(19) United States
(12) Patent Application Publication
(10) Pub. No.: US 2018/0000704 A1
(43) Pub. Date: Jan. 4, 2018

(54) USES OF CO-CRYSTALS

(71) Applicant: University of Strathclyde, Glasgow (GB)

(72) Inventors: Iain OSWALD, Glasgow (GB); Amit DELORI, Glasgow (GB)

(21) Appl. No.: 15/547,457

(22) PCT Filed: Feb. 1, 2016
(86) PCT No.: PCT/GB2016/050219

§ 371 Date: Jul. 28, 2017

(30) Foreign Application Priority Data
Jan. 30, 2015 (GB) 1501583.7

(51) Int. Cl. A61K 8/362; C09B 67/48; A61Q 5/10; A61K 8/365; A61K 8/41; A61K 8/49; A61K 8/46; C09B 67/22; A61Q 5/06

(52) U.S. Cl. CPC A61K 8/362; C09B 67/0033; C09B 67/0025; A61Q 5/10; A61Q 5/065; A61K 8/411; A61K 8/498; A61K 8/492; A61K 8/46; A61K 8/365; A61K 2800/56

(57) ABSTRACT

The present invention discloses new uses of coloured co-crystals and associated salts formed by co-crystallization reactions in the dye, ink and paint industries. The present invention also provides novel co-crystals for use in, for example, the dye, ink and/or paint industries.

### FIELD OF THE INVENTION

The present invention discloses new uses of coloured co-crystals and associated salts formed by co-crystallization reactions in the dye, ink and paint industries. The present invention also provides novel co-crystals for use in, for example, the dye, ink and/or paint industries.

### BACKGROUND TO THE INVENTION

Coloured compounds are of great interest to the multi-billion dollar colour industry (such as paint, ink and dye industry). All coloured molecules have one thing in common that they all absorb in the visible region. It is due to the fact that the HOMO-LUMO energy gap (HLEG) of these molecules lies in the visible region. In contrast HLEG of colourless molecules is usually much higher than that required to absorb in the visible region. Scientists discovered that HLEG of molecules can be decreased by introduction of conjugation and in fact conjugation has long being used as a strategy for the design and synthesis of coloured molecules. Unfortunately, design and synthesis of conjugated molecules involves complex synthetic pathways, which also add to their cost.

In a recent report from Yan et al. (D. Yan, A. Delori, G. O. Lloyd, T. Fri\check{s}\check{c}ić, G. M. Day, W. Jones, J. Lu, M. Wei, D. G. Evans, X. Duan, Angew. Chem., Int. Ed. 2011, 50, 12483-12486), the authors reported that they were able to tune the HLEG by co-crystallization and used it to change the fluorescent properties of the materials.

Co-crystallization reactions are known for their ease. In these reactions different molecule recognize each other by a process known as self-assembly and are simply carried out by mixing 2 or more molecules in a suitable medium.

### SUMMARY OF THE INVENTION

The present invention is based upon work carried out by the present inventors in relation to the development of coloured co-crystals for use in the paints, inks and/or dyes industry, for example.

In a first aspect there is provided use of a coloured co-crystal formed from a first and a second component (herein referred to as first and second coformers) in the manufacture of a dye, ink and/or paint, especially as a dye, such as a hair dye.

Herein we discuss the provision of co-crystals for use in, for example, the dye, ink and/or paint industries. In this disclosure we use the term ‘co-crystal’ as defined by Aitipamula et al. ‘cocrystals are solids that are crystalline single phase materials composed of two or more different molecular and/or ionic compounds generally in a stoichiometric ratio’ thereby including salt forms and/or solvates. (S. Aitipamula, et al, Crystal Growth & Design, 2012, 12, 4290-4291).

The co-crystals of the present invention may contain common dye, pigments and fluorescent molecules as one of the components, such as: N-Phenyl-Para-Phenylene Diamine, 1,5-Naphthalenediamine, 4-lodoaniline, 1-anilinonaphthalene-8-sulfonic acid, 1,3-Dichloro-7-hydroxy-9,9-dimethyl-2(9H)-acridinone, 1,4-bis-p-cyanostyrylbenzene, 2-((E)-4-((E)-4-cyanostyryl)styryl)benzonitrile, 2,2′-((1E,1′E)-1,4-phenylenebis(ethene-2,1-diyl))dibenzonitrile, 2-Methylbenzoxazole, 2,7-dichlorofluorescein, 2′,7′-bis-(2-carboxyethyl)-5-(and-6)-carboxyluorescein 2,5-diphenyloxazole, 4-Dimethylamino-4′-nitrostilbene, 5(6)-Carboxyfluorescein, 5(6)-Carboxynaphtofluorescein, 5(6)-Carboxytetramethylrhodamine B, 5-(and-6)-carboxy-2′,7′-dichlorofluorescein, 5-(N-hexadecanoyl)aminoeosin, 5-Chloromethylfluorescein, 5-carboxyfluorescein, 6,8-difluoro-7-hydroxy-4-methylcoumarin, 6-carboxyrhodamine 6G, 7-hydroxy-4-methylcoumarin, Acridine (orange and yellow), Alexa Fluor, Alcian yellow GXS, Alizarin, Alizarin red S, Alizarin yellow GG, Alizarin yellow R, Anthoxanthin, Arylide yellow, ATTO, Auramine O, Azophloxin, Azo compound, Bilin, Bismarck, Bistre, Bone char, brown R, Bismarck brown Y, Brilliant cresyl blue, Calcein, Caput mortuum, Carmine, Chrysoidine R, Chrysoidine Y, Coumarin, Congo red, Crimson, Crystal violet, Doxorubicin, Diarylide pigment, Dragon's blood, Fluorescein, Fuchsin acid, epicocconone, Gamboge, Gentian violet, Indian yellow, Indigo dye, Janus green, Lissamine fast yellow, Marina Blue, Martius yellow, Meldola blue, Metanil yellow, Methyl orange, Methyl red, Methylene Blue, Monobromobimane, Monochlorobimane, Naphthalene black 12B, Naphthofluorescein, Naphthol green B, Naphthol yellow S, Naphthol Red, nile blue, nile red, Oxazin, Ommochrome, Orange G, Perinone, Phthalocyanine Blue BN, Phthalocyanine Green G, Pigment Yellow (10,16, 81, 83), Piroxicam, Quinacridone, Riboflavin, Rose bengal, Rose madder, Rylene dye, Sepia (color), Sudan II, Sulforhodamine, Titan yellow, Tyrian purple, Tropaeolin O, Tropaeolin OO, Tropaeolin OOO, Victoria blue 4R, Victoria blue B, Victoria blue R, Xylene cyanol FF), but not only limited to them.

Typically the second component or further components (co-former(s)) may be chosen by considering the synthon complementarity. The only requirement for such co-formers is the presence of hydrogen bond donors and acceptors functionalities. Due to this co-formers can be chosen from a range of molecules such as those containing carboxylic acid, alcohol, amines, aldehydes, nitro, nitroso, ketones, ethers, esters, amides, acetals, ketals, imide, nitrile, isonitrile, halo, acylhalide, nitroso, pyridine, sulfoxide, sulfinic acid, sulphonic acids, cyanates, isocyanates, thiols, thial, thione, thiocyanates, isothiocyanates, semicarbazones, thiosemicarbazones, ureas, thioureas, amides, phosphonic acids, phosphine, phosphate, phosphodiester, boronic acids, boronic esters, borinic acids and borinic esters functionalities.

Examples of some suitable co-formers include aliphatic and aromatic mono, di or polycarboxylic acids and alcohols of variable chain lengths (e.g acetic acid, oxalic acid, malonic acid, maleic acid, lactic acid, tartaric acid, citric acid, fumaric acid, succinic acid, acetylenedicarboxylic acid, mesaconic acid, trans-acotinic acid, thiodiglycolic acid, diglycolic acid, glutaric acid, adipic acid, hexanoic acid, pimelic acid, suberic acid, octanoic acid, azelaic acid, sebacic acid, decanoic acid, undecanedioic, dodecanedioic, oleic acid, arachidic acid, stearic acid, palmitic acid, erucic acid, arachidonic acid, linoleic acid, linolenic acid, phthalic acid, isophthalic acid, terephthalic acid, benzenetricarboxylic acids, benzenetetrracarboxylic acids, benzenepentacarboxylic acids, benzenehexacarboxylic acid, disubstituted benzoic acids e.g. 3,5 dinitrobenzoic acid, phenylenediacetic acids, phenols, benzenediol, benzenetriols, benzenetetraols, benzenepentaols, benzenehexaols, aliphatic alcohols containg one of more hydroxyl groups). Co-formers may also contain more than one functional groups e.g. amino-benzoic acids, nitrobenzoic acids, halo-benzoic acids, phosphonobenzoic acids, formylbenzoic acids, cyanobenzoic acids, isocyanobenzoic acids, sulfobenzoic acids, boronobenzoic acids, nicotinic acid, isonicotinic acid, nicotinamide, isonicotinamide etc.

Examples of suitable alcohols and/or ketones include alcohols and/or ketones which are understood to be the pigmented compounds of natural dyes extracted from plants, animals and minerals. Such suitable alcohols and ketones include flavonoids, such as flavones, flavanols, isoflavones, chalcones and catechins; terenoids and isoprenoids; naphthoquinones and anthraquinones; and alkanoids. In a preferred embodiment suitable alcohols and/or ketones are naphthoquinones and anthraquinones, such as naphthoquinone, juglone, lawsone, alkannin, anthraquinone or alizarin. A particularly preferred naphthaquinone for use in the present invention is Lawsone.

In an embodiment of the present invention a coloured co-crystal may be formed from p-phenylenediamine (PPD) or a derivative thereof in combination with a second component as defined herein above.

As well as PPD itself, derivatives such as p-toluenediamine; 2-chloro-p-phenylenediamine; N-phenyl-p-phenylenediamine; N-2-methoxyethyl-p-phenylenediamine; N,N-bis-hydroxyethyl-p-phenylenediamine; 2-hydroxymethyl-p-phenylenediamine; 2-hydroxyethyl-p-phenylenediamine; 4,4′-diaminodiphenylamine; 2,6-dimethyl-p-phenylenediamine; 2-isopropyl-p-phenylenediamine; N-(2-hydroxypropyl)-p-phenylenediamine; 2-propyl-p-phenylenediamine; 1,3-bis-(N-hydroxyethyl)-N-(4-aminophenyl)amino)-2-propanol; and 2-methyl-4-dimethylaminoaniline may be used instead.

Particularly preferred coloured co-crystals formed in accordance with the present invention for use as hair dyes include PPD, or its derivatives such as N-phenyl-p-phenylenediamine, or 1,5-napthalenediamine cocrystallized with an acid defined above, such as fumaric (FA), succinic (SA), glycolic, malonic, glutaric, thiodiglycolic (TDGA), adipic (AA), suberic (SubA) and azelaic (AzeA), sebacic (SebA), 1,3-phenylenediacetic, (1,3-PDAA), 1,4-phenylenediacetic, (1,4-PDAA) and trimesic (TMA) acids. Specific coloured co-crystals are disclosed in the examples section, but this should not be construed as limiting.

It is to be understood that the term “coloured” is intended to mean that the co-crystals of the present invention display a colour within the visible spectrum, typically in the range 380-760 nm in wavelength. In certain embodiments of the present invention, the coloured co-crystals of the present invention may appear yellow, orange, red, brown, black or various shades in between.

Co-crystallisation reactions are known in the art for their ease. In these reactions different molecules recognize each other by a process known as self-assembly and may simply be carried out by mixing 2 or more molecules in a suitable medium. These reactions can also be carried out by various other ways also such as by grinding (A. Delori, T. Fri\check{s}\check{c}ić, W. Jones, CrystEngComm 2012, 14, 2350-2362), liquid-assisted grinding (LAG) (A. Delori, W. Jones, CrystEngComm 2011, 13, 6315-6318) melting (Katharina Fucke et al New J. Chem., 2012,36, 1969-1977), evaporation (Katharina Fucke et al New J. Chem., 2012,36, 1969-1977), fast evaporation using rotavapor (Partha Pratim Bag et al CrystEngComm, 2011,13, 5650-5652), vapour diffusion (R. P. Rastogi, et al J. Phys. Chem., 1962, 66, 2707-2708), precipitation (Katharina Fucke, et al New J. Chem., 2012,36, 1969-1977) and hot-melt extrusion (R. S. Dhumal, et al Pharm. Res. 2010, 27, 2725-2733), freeze drying (M D Eddleston, et al—Crystal Growth & Design (2013) 13,4599), anti-solvent precipitation (A Delori, et al, CrystEngComm 2014, DOI: 10.1039/C4CE00211C etc. Recently, Zhao et al. (L. Zhao, et al, CrystEngComm 2014) demonstrated that co-crystallisation reactions can also be carried out by a continuous crystallization method for scalability. Any of the aforementioned co-crystallisation methods may be employed in connection with the present invention in order to provide the co-crystals of the present invention.

In one embodiment, the co-crystals of the present invention may find application in the preparation of hair dyes and hence the co-crystals of the present invention may be formulated into hair dye compositions for dying hair. Hair which may be treated in accordance with the present invention, by applying a hair dye composition comprising one or more co-crystals of the present invention to hair, in order to dye the hair. The term “hair” is intended to include both natural animal, typically human hair and synthetic hair which may be part of a wig.

PPD is widely used and an important precursor of hair dyes and is used in most permanent hair dyes. PPD-based hair dyes are usually marketed in their leuco (colourless) form and users are required to synthesize the coloured end product themselves prior to use, which can of course be problematic. PPD-based hair dye kits known in the art usually contain 5 components (2 tubes, mixing bowl, gloves and a conditioner). The main ingredients of the PPD-based hair dyes are packed in 2 separate tubes. One tube contains PPD, ammonia and a coupler and the other tube contains H₂O₂. Mixing of contents of these 2 tubes (in the mixing bowl provided with the hair dye kit), results in the formation of bigger conjugated molecules, which are coloured. Due to the presence of strong hydrogen bond donors and acceptors these bigger molecules bind strongly with the hairs and hence are used as hair dyes. In these hair dye formulations ammonia is added to open up the pores of the hairs for the better penetration of the dye molecules in the hairs. Customers are advised to wash their dyed hairs, with the hair conditioner, provided with the kit, for the longer lasting hair colours. It is believed that these conditioners again reduce the size of the pores in the hairs and help in trapping the dye molecules for longer time. The companies strongly recommend the customers to wear the gloves during synthesis of hair dyes.

There are two major concerns associated with these hair dye formulations. The major concern is the safety of these hair dyes, especially these dyes have been queried for their carcinogenic nature. The matter was recently thoroughly investigated by the Scientific Committee on Consumer Safety (SCCS). In their 75 page report, they revealed that though these dyes are safe to be applied on hairs, the chances of tumours increases if these dyes come in topical contact with the skin (http://ec.europa.eu/health/scientific_committees/consumer_safety/docs/sccs_o_094.pdf.). Unfortunately, there is always a danger of contact of these dyes with the skin (especially with the skin of the skull) during dying of hair. Interestingly, the committee revealed that topical contact of PPD alone with skin doesn't pose such carcinogenic risks. However, some end-products of synthetic technology based PPD hair dyes were found to be carcinogenic. The hair dyes of the present invention avoids the synthesis of such new molecules, avoiding potential risks.

The second concern associated with these hair dyes is that they use ammonia. A study has revealed that long term use of ammonia on hairs can permanently damage them. With this in mind, the hair dying industry has developed ammonia free versions of the PPD based dyes, but unfortunately these ammonia free versions of PPD based dyes are not efficient on difficult to dye hairs, such as grey. The present inventors have observed that the co-crystals as defined herein, when used as hair dyes work well even without ammonia. Thus, in preferred embodiments of the present invention the hair dye compositions and kits do not necessarily require ammonia.

The constituents of the co-crystals may be separated for mixing by the intended user, immediately prior to use, for example, or the coloured co-crystals may already be in a form for use by the intended user.

The hair dye compositions of the present invention may be pre-formulated into a solution, cream, lotion, gel, emulsion, or the like. The hair dye compositions of the present invention may further comprise other components such as wetting agents or emulsifying agents from the categories of anionic or non-ionic surfactants, such as sulfates of fatty alcohols, alkanolamides of fatty alcohols, alkyl sulfonates, alkylbenzene sulfonates, oxyethylated fatty alcohols, oxyethylated nonylphenols; furthermore thickeners such a fatty alcohols, starch, cellulose derivatives, paraffin oil and fatty acids, as well as hair-care substances such as lanolin derivatives, cholesterol and pantothenic acid, may be formulated into the compositions of the invention.

As an example, if formulated as a lotion, the compositions of the invention may contain organic solvents to assist in dissolving the coloured co-crystals. Accordingly, the organic solvent can be present in any suitable amount, preferably, about 1% to about 15%. Typically useful solvents include alcohols containing up to three carbon atoms such as methanol, ethanol and isopropanol, polyhydroxy alcohols such as propylene or hexylene glycol and lower alkyl ethers thereof, such as ethoxy ethers.

In addition, the hair dyeing compositions in accordance with the present invention may optionally contain conventionally-used adjuvants and cosmetic additives, or mixtures thereof, to achieve the final formulations. Examples of such additives include, but are not limited to, anti-oxidants, e.g., ascorbic acid, erythoboric acid, or sodium sulfite, to inhibit premature oxidizing; oxidizing agents; fragrances and/or perfume oils; chelating agents; emulsifiers; coloring agents; thickeners; organic solvents; opacifying agents; dispersing agents; sequestering agents; hair-care substances; humectants; and anti-microbials, and others. The list of optional ingredients is not intended as limiting. Other suitable adjuvants for inclusion in the hair dye compositions of the invention are disclosed, for example, in Zviak, The Science of Hair Care (1986) and in Balsam and Sagarin, Cosmetics: Science and Technology, Vol. 2, Second Edition, (1972).

Thickeners that may be used in the compositions of the present invention include a variety of fatty acid soaps and associative polymeric thickeners. The fatty acid soaps are alkaline metal salts or alkanolamine salts of fatty acids with C₁₀-C₁₆ alkyl side chains. The preferred fatty acids include oleic acid, myristic acid and lauric acid, which are generally present in the compositions of the invention at about 0.5% to about 20%, preferably about 1% to about 10%. Associative thickeners are polymers that can thicken solutions at low concentrations. Among the associative thickeners that are useful in the compositions of the present invention are acrylates copolymer (sold by Rohm and Haas under the tradename Aculyn-33), ceteareth-20 acrylates/steareth-20 methacrylate copolymer (sold by Rohm and Haas under the Trade Name Aculyn-22), acrylates/steareth-20 itaconate copolymer and acrylates/ceteth-20 itaconate copolymer. Another class of associative thickeners that is useful in the compositions of the present invention include the copolymers of polyurethane and polyethylene glycol or polyether urethanes. One such material is sold by Rohm and Haas under the tradename Aculyn-44. The associative polymeric thickeners are generally present in the compositions of the invention at about 0.1% to about 10%, preferably, about 0.5% to about 5%.

The compositions of the invention may include a typical anionic, cationic, nonionic or amphoteric surfactant. The anionic surfactants include the variety of alkyl sulfates, alkylether sulfates, alkyl sulfonates, alkyl sulfosuccinates and N-acyl sarcosinates. The commonly-used anionic surfactants are sodium and ammonium lauryl sulfates, sodium and ammonium laureth sulfate and alpha olefin sulfonates. Anionic surfactants are generally present in the compositions of the present invention at about 0.1% to about 15%, preferably, about 0.5% to about 10%.

The nonionic surfactants that can be used in the present invention include the wide variety of ethoxylated alcohols, nonoxynols, alkanolamides, alkyl stearates, alkyl palmitates and alkylpolyglucosides. Examples of the commonly-used nonionic surfactants are cetyl alcohol, stearyl alcohol, oleyl alcohol; the various types of ethoxylated alkylphenols; lauramide diethanolamide (DEA); lauramide monoethanolamide (MEA); isopropyl palmitate, isopropyl stearate and decylpolyglucoside. Nonionic surfactants are generally present in the compositions of the present invention at about 0.1% to about 15%, preferably, about 0.5% to about 10%.

The compositions in accordance with the present invention may also contain one or more quaternary ammonium compounds that provide hair conditioning effects. The quaternary ammonium compounds can be monomeric or polymeric quaternary ammonium compounds. Non-limiting examples of such compounds include cetyltrimonium chloride, stearyl trimonium chloride, benzalkonium chloride, behentrimonium chloride and a variety of polyquaterniums. The quaternary ammonium compounds are generally present in the compositions of the present invention at about 0.1% to about 10%, preferably, about 0.5% to about 5%.

Amphoteric surfactants that can be incorporated in the compositions of the present invention belong to the class of surface active chemicals that possess a positive and a negative charge in the same molecule and behave as a cation, an anion, or both, depending upon the pH of the medium and the nature of the amphoteric molecule. In general, the positive charge is located on a nitrogen, while the negative charge is carried by a carboxyl or sulfonate group. There are a large number of amphoteric surfactants that are suitable for use in the present invention, including, for example, the well-known betaines, sultaines, glycinates and propionates.

The selection of the amphoteric surfactant or mixture of surfactants for use in the present compositions and methods is not critical. The surfactant may be selected from among those suggested above, or from any of a number of other known amphoteric surfactants. The amount of amphoteric surfactant in the compositions of the present invention is normally from about 0.5% to about 15%, preferably, about 2% to about 10%.

Depending on the final formulated preparation, the compositions in accordance with invention may be weakly acidic, neutral or alkaline. In particular, the pH of the prepared compositions can range from about 5 to about 11. Preferred is a pH range of about 8 to 10. Any of a wide variety of alkaline reagents can be used to adjust the pH of the hair coloring compositions. Such alkaline reagents include ammonium hydroxide, potassium or calcium hydroxide, sodium or potassium carbonate, sodium phosphate, sodium silicate, guanidine hydroxide, or any one of the alkylamines or alkanolamines, for example, ethylamine, triethylamine, trihydroxymethylamino amine, ethanolamine, diethanolamine, aminomethyl propanol, aminomethyl propanediol and the like. The preferred alkaline reagents are ammonium hydroxide, sodium carbonate and ethanolamine. With the reagents listed above, the selected pH will generally be achieved if the composition contains an alkaline agent in an amount from about 0.1% to about 15%, preferably, about 0.5% to about 5%.

Typically, the hair dye compositions are intended to provide a permanent or semi-permanent dying of the hair. The term “permanent or semi-permanent” means the dye does not readily wash out of the hair with ordinary shampoos, for period of at least a week, preferably 2 weeks, 4 weeks, 2 months, 6 months or more. At the end of hair dying application (e.g., approximately 5 to 45 minutes, preferably, approximately 10 to 30 minutes), the composition/excess dye is washed from the hair with an ordinary water rinse, optionally followed by a shampoo. The application temperature is typically in the range of about 15° C. to 50° C.

The hair dyeing compounds in accordance with the invention will offer a wide range of varying color tints depending upon the type and composition of the colorant constituents. In accordance with the present invention, the hair dye compositions of the present invention may comprise one or more coloured co-crystals of the present invention.

Particularly preferred hair dye compositions comprise coloured co-crystals formed in accordance with the present invention include PPD, or derivatives such as N-phenyl-p-phenylenediamine (NP-PPD), or 1,5-napthalenediamine (1,5-DAN), wherein PPD, NP-PPD or 1,5-DAN is cocrystallized with an acid defined above, such as fumaric (FA), succinic (SA), glycolic, malonic, glutaric, thiodiglycolic (TDGA), adipic (AA), suberic (SubA) and azelaic (AzeA), sebacic (SebA), 1,3-phenylenediacetic, (1,3-PDAA), 1,4-phenylenediacetic, (1,4-PDAA) and trimesic (TMA) acids. Additionally some already known coloured crystals were tested for their dying potential such as cocrystals of fluorescein and acridine and methanol solvate of 4-iodoanailine and 3,5 dinitrobenzoic acid. Specific coloured hair dyes are disclosed in the examples section, but this should not be construed as limiting.

The compositions of this invention may be provided in a kit or packaged form ready for application by the user, either professional or consumer, to initiate the dying process. The kit provided in accordance with this invention comprises containers for housing the hair dye compositions of the invention. Unlike in prior art hair dye compositions, the hair dye compositions of the present invention may not require to be mixed by the user prior to application to hair and may already be in a coloured ready prepared form.

In accordance with the present invention there is provided a method of dying hair, the method comprising applying a hair dye composition or coloured co-crystal(s) of the present invention to hair to be colored and allowing it to remain in contact with the hair until the desired hair color has been attained, after which time the composition is removed from the hair as described above. Typically the contact time with the hair to be dyed may be in the region of 5 mins to 1 hour.

In order to facilitate dyeing of hairs, a swelling agent, such as water, a solvent, amine, organic and inorganic acid, salt, urea, formamide, may be added for a period of time, for example, 1-10 min, such as 2-7 min, before applying the dye, in order to increase hair pore size.

In one embodiment, the co-crystals of the present invention may find application in the preparation of inks. Thus, in a further aspect, the present invention provides a coloured ink comprising one or more coloured co-crystal(s) as described herein. The inks may comprise a single type of co-crystal, or may comprise more than one type of co-crystal. Preferred co-crystals are formed between PPD and the identified organic acids.

Advantageously, some of the co-crystals of the present invention when prepared as inks display significant water resistance when applied to paper. The inks of the present invention may be removed from paper by application of a solvent, such as an alcohol containing up to three carbon atoms such as methanol, ethanol and isopropanol, polyhydroxy alcohols such as propylene or hexylene glycol and lower alkyl ethers thereof, such as ethoxy ethers.

The coloured co-crystals when used to prepare inks, avoids the need to generate coloured molecules by complex synthetic pathways and hence has the potential to reduce the cost of inks dramatically.

Along with coloured co-crystals the inks of the present invention may contain other common ink ingredients such as (1) solvent, (2) binders/resins, (3) humectants (retard premature drying), (4) antifoaming/defoaming agents, (5) Permeation promoter, (6) Anti-mould agent, (7) Surface tension controller (8) pH modifiers (9) dispersants, and/or (9) wetting agents

Solvent for ink: Any suitable organic solvent can be used, preferably volatile organic solvents, e.g., solvents having a boiling point of less than 100° C., particularly less than 85° C. In an embodiment, the one or more organic solvents may be selected from the group consisting of ketones, alcohols, esters, and ethers, e.g., glycol ethers. Any suitable ketone can be used, for example, a lower alkyl ketone such as acetone or methyl ethyl ketone, or a cyclic ketone such as cyclohexanone. Examples of esters include alkyl esters such as ethyl acetate, ethyl propionate, ethyl lactate, propyl acetate, and butyl acetate. Examples of alcohols include lower alkyl alcohols such as methanol, ethanol, propanol, isopropanol, and butanol. Examples of glycol ethers include ethylene glycol monomethyl ether, diethylene glycol monomethyl ether, diethylene glycol monobutyl ether, triethylene glycol monomethyl ether, triethylene glycol monobutyl ether, dipropylene glycol monomethyl ether, dipropylene glycol monobutyl ether, tripropylene glycol monomethyl ether, and tripropylene glycol monobutyl ether. A mixture of one, two, three, or more organic solvents can be employed.

The organic solvent or a mixture of organic solvents can be present in any suitable amount, for example, in an amount greater than 50% by weight, preferably from about 60% to about 90%, more preferably from about 70% to about 80%, by weight of the embodiments of the ink composition. In a specific embodiment, the ink composition includes a mixture of a more volatile solvent (e.g., a solvent having a boiling point about 56° C.) and a less volatile solvent (e.g., a solvent having a boiling point about 145° C.), for example, a mixture of a more volatile ketone solvent and a less volatile glycol ether solvent. Thus, for example, the ink composition can include a mixture of acetone and propyleneglycol methyl ether.

Where a mixture of solvents is used, the mixture can contain any suitable proportion of the solvents. For example, in a mixture containing one or more volatile solvents (e.g., boiling point less than 85° C.) and one or more less volatile solvents (e.g., boiling point less than 200° C.), the weight ratio of the more volatile solvent or solvents to the less volatile solvent or solvents can be about 90/10, about 80/20, about 70/30, about 60/40, about 50/50, about 40/60, or about 30/70, preferably about 90/10, about 80/20, or about 70/30, or any ratio there-between.

Binders for ink: The ink composition of the invention contains one or more solvent-soluble binder resins, i.e., resins that are soluble in the organic solvent or the mixture of organic solvents. Any suitable solvent-soluble binder resin or resins can be employed. Examples of solvent-soluble binder resins include cellulosic resins, acrylic resins, styrene-acrylic resins, styrene-maleic anhydride resins, vinyl resins such as polyvinyl chloride, polyvinyl acetate or polyvinyl alcohol resins, rosin resins, silicone resins, phenolic resins, novolac resins, ketone resins, aldehyde resins, polyester resins, polyamide resins, polyimide resins, terpene resins, alkyd resins, polyurethane resins, ketal resins, epoxy resins, chlorinated rubber, shellac, and Saran resin.

Any suitable cellulosic resin can be employed, for example, a cellulose ester or an alkylcellulose. Cellulose ester is cellulose some or all of whose hydroxyl groups have been modified to have an ester function or mixed ester functions, e.g., by one or more ester groups wherein the ester group has 2-8 carbon atoms, preferably 2-5 carbon atoms. Examples of cellulose ester include cellulose mixed esters such as acetate butyrate and cellulose acetate propionate. An example of a suitable cellulose ester is cellulose acetate butyrate available commercially as CAB 551-0.01 from Eastman Chemical, Kingsport, Tenn. The alkylcellulose is cellulose some or all of whose hydroxyl groups have been modified to contain an alkyl group of 1-8 carbon atoms, preferably 2-4 carbon atoms, e.g., ethylcellulose. Nitrocellulose can also be employed as a cellulosic resin.

Any suitable acrylic resin or styrene-acrylic resin can be used. In the styrene-acrylic resin, the acrylic monomer can be an acrylic ester or acrylic acid, for example a copolymer of styrene and acrylic monomer having an acid number of from 0 to about 200, preferably from about 10 to about 100. Examples of solvent-soluble styrene-acrylic resins are JONCRYL™ 586 and JONCRYL 611 resins available from S.C. Johnson Co., Racine, Wis. In a specific embodiment, the ink composition includes cellulose acetate butyrate and styrene-acrylic resin as solvent-soluble binder resins.

The solvent-soluble binder resin or resins can be present in the ink composition in any suitable amount, for example, in an amount of about 3% or more, such as from about 5 to about 25% or more, preferably from about 10 to about 20%, and more preferably from about 12 to about 16% by weight of the ink composition. For example, where a mixture of two resins are employed, the resins can be present in any suitable proportion, e.g., about 20/80, about 25/75, about 30/70, about 40/60, or about 50/50 of the two resins by weight, or any proportion there-between.

Humectants: The amount of humectant used is determined by the properties of the ink and may range from 1-30%, preferably 5-15%, by weight, based on the total weight of the ink. Examples of commonly used humectants useful in forming the ink are: glycols, polyethylene glycols, glycerol, ethanolamine, diethanolamine, alcohols, and pyrrolidones. Other humectants known in the art may be used as well. The drying inhibitor is used preferably with an aim of preventing clogging due to drying of the ink. The drying inhibitor is preferably a water soluble organic solvent having a vapor pressure lower than that of water. Specific examples include polyhydric alcohols typically represented by ethylene glycol, propylene glycol, diethylene glycol, polyethylene glycol, thiodiglycol, dithiodiglycol, 2-methyl-1,3-propanediol, 1,2,6-hexanetriol, acetylene glycol derivatives, glycerine, and trimethylol propane, lower alkyl ethers of polyhydric alcohols such as ethylene glycol monomethyl(or ethyl) ether, diethylene glycol monomethyl (or ethyl) ether, and polyethylene glycol monoethyl (or butyl) ether, heterocyclic rings such as 2-pyrrolidone, N-methyl-2-pyrrolidone, 1,3- dimethyl-2-imidazolidinone, and N-ethyl morpholine, sulfur-containing compounds such as sulfolane, dimethyl sulfoxide, and 3-sulfolene, poly-functional compounds such as diacetone alcohol and diethanol amine, and urea derivatives. Among them, polyhydric alcohols such as glycerine and diethylene glycol are more preferred. The drying inhibitors can be used each alone or two or more of them may be used in combination. The drying inhibitor is preferably contained by 10 to 50% by weight in the ink.

Defoaming agents: The amount of defoaming agent in the ink will typically range from 0-0.5% by weight, based on the total weight of the ink. Defoaming agents useful in forming aqueous dispersions of pigments are well known in the art and commercially available examples include Surfynol 104H and Surfynol DF-37 (Air Products, Allentown, Pa.). As the defoamer, fluoro type and silicone type compounds or chelating agents typically represented by EDTA may also be used optionally.

Permeation promoter: The permeation promoter is used preferably with an aim of effectively permeating the ink to paper. As the permeation promoter, alcohols such as ethanol, isopropanol, butanol, di(tri)ethylene glycol monobutyl ether, and 1,2-hexanediol and nonionic surfactants such as sodium lauryl sulphate and sodium oleate can be used. They are usually provide a sufficient effect when incorporated by 5 to 30% by weight in the ink and are used preferably within a range of the addition amount not causing blur of printing and print-through.

Anti-mould agent: The anti-mould agent includes, for example, sodium dihydro acetate, sodium benzoate, sodium pyridinethion-1 -oxide, ethyl p-hydroxy benzoate ester, 1,2-benzoixothiazolin-3-one and salts thereof. They are preferably used by from 0.02 to 1.00% by weight in the ink.

Surface tension controller: The surface tension controller includes cationic or anionic surfactant. Examples of the surfactant are preferably anionic surfactant such as fatty acid salts, alkyl sulfate ester salts, alkylbenzene sulfonate salts, alkylnaphthalene sulfonate salts, dialkylsulfosuccinate salts, alkylphosphate ester salts, naphthalene sulfonic acid formalin condensate, and polyoxyethylene alkyl sulfate ester salts, and nonionic surfactant such as polyoxyethylene alkyl ether, polyoxyethylene alkyl allyl ether, polyoxyethylene fatty acid ester, sorbitan fatty acid ester, polyoxyethylene sorbitan fatty acid ester, polyoxyethylene alkylamine, glycerine fatty acid ester, and oxyethylene oxypropylene block copolymer. Further, SURFYNOLS (manufactured by Air Products & Chemicals Co.) as the acetylene type polyoxyethylene oxide surfactant is also used preferably. Further, amino oxide type amphoteric surfactant such as N,N-dimethyl-N-alkylamine oxide is also preferred. Further, those referred to as surfactants described in JP-A No. 59-157630, pp (37) to (38), and Research Disclosure No. 308119 (1989) can also be used.

pH controller: As the pH controller, the neutralizing agent (organic base or inorganic alkali) can be used. The pH controller is added with an aim of improving the store stability of the ink for use in an ink jet, for example, so as to control the pH of the ink for use in ink jet to 6 to 10 and, more preferably, 7 to 10.

Dispersants: Dispersants stabilise the inks. Two classes of compounds are used for this purpose: surfactants and polymers. These compounds adsorb to the co-crystal particles and form a coating of varying composition and thickness. The resulting modified particle surfaces either attract or repel each other—leading to flocculation or stabilisation, respectively. Flocculation hampers dispersion, and stabilising forces are essential to prevent the fine particles of co-crystals from settling. The size and shape of the co-crystal particles dictates the colour intensity, shade and light fastness. Surfactants and polymers are used as dispersants such as Nitrocellulose based polymers, polyacrylate homopolymers and copolymers, polyurethanes and polyesters, Sodium dodecyl sulfate, Cetyl trimethyl ammonium bromide, Dodecyl octaethyleneglycol monoether, N-n-Dodecyl-N,N-dimethyl betaine

### DETAILED DESCRIPTION

The present invention will now be further described by way of example and with reference to the figures which show:

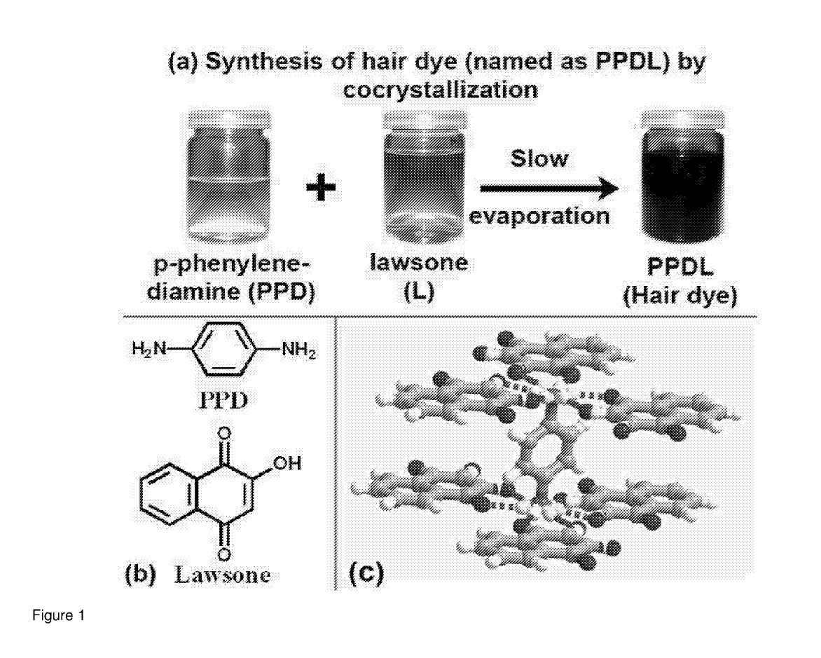

FIG. 1 shows (a) Colour change observed during the co-crystallisation reaction between (b) PPD and L and (c) Crystal structure of salt between PPD and L;

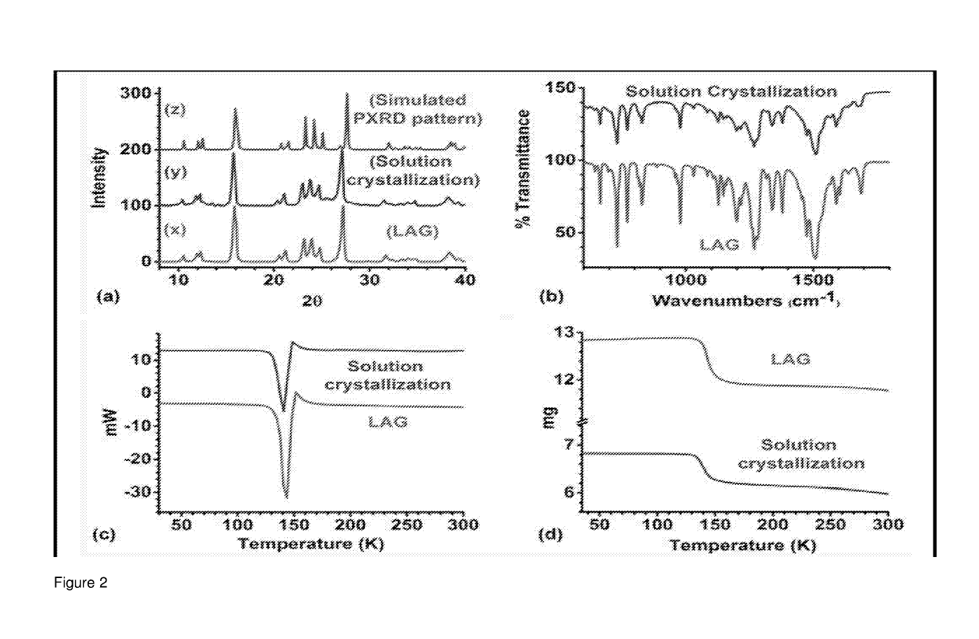

FIG. 2 shows (a) Comparision of PXRD patterns of phase pure materials formed between PPD and L by LAG (x), solution crystallization (y), with the simulated pattern obtained from their crystal structure (z). (b-d) Comparision of IR, DSC and TGA spectra of the materials obtained via solution and LAG;

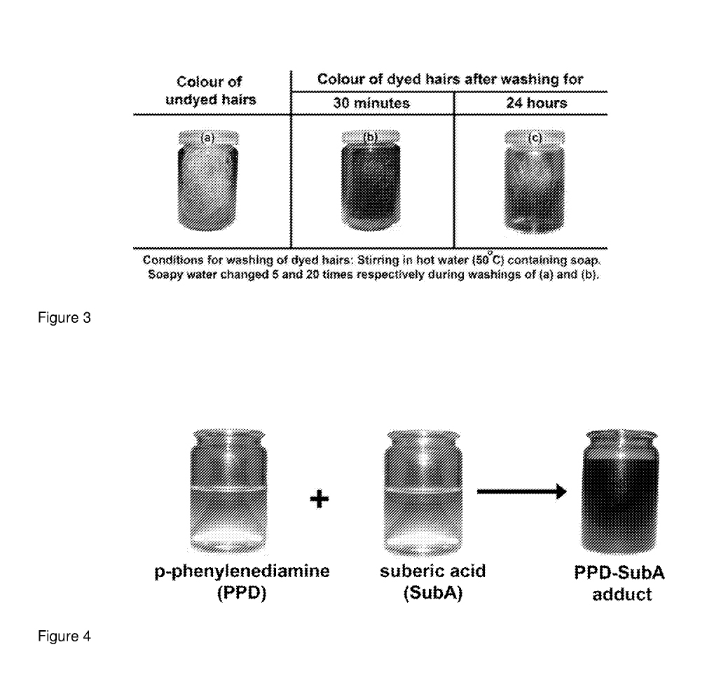

FIG. 3 shows (a) Undyed hairs. Dyed hairs (with PPDL dye) after washing continuously in hot soapy solution for (b) 30 minutes and (c) 24 hours;

FIG. 4 shows the colour change observed during co-crystallisation of PPD with suberic acid;

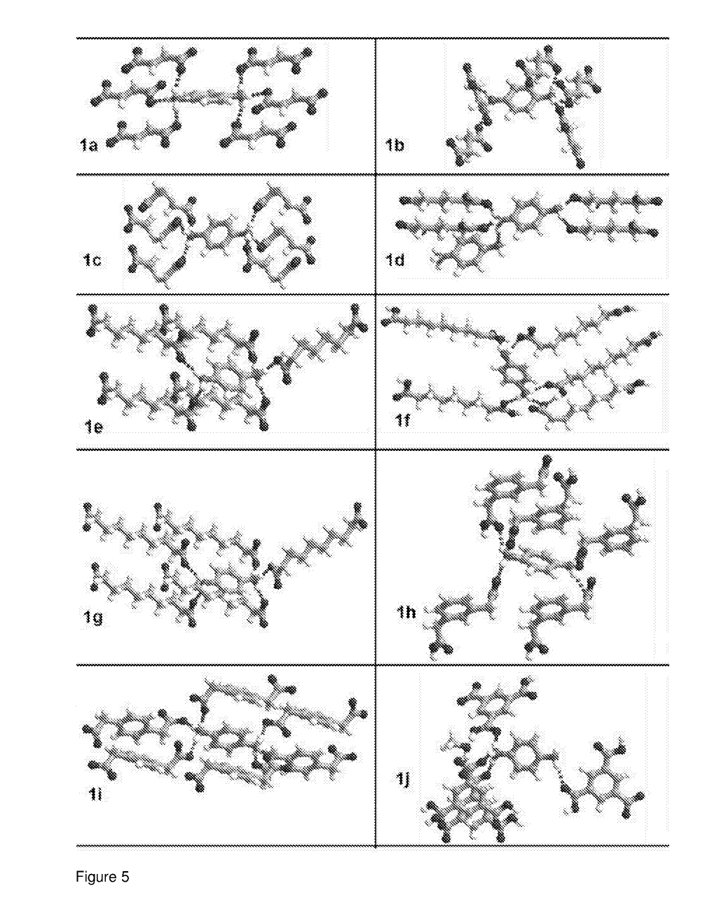

FIG. 5 shows the Recognition pattern observed in the crystal structures of (a) PPD-FA, (b) PPD-SA, (c) PPD-TDGA, (d) PPD-AA, (e) PPD-SubA, (f) PPD-AzeA, (g) PPD-SebA (h) PPD-1,3-PDAA (i) PPD-1,4-PDAA (j) PPD-TMA

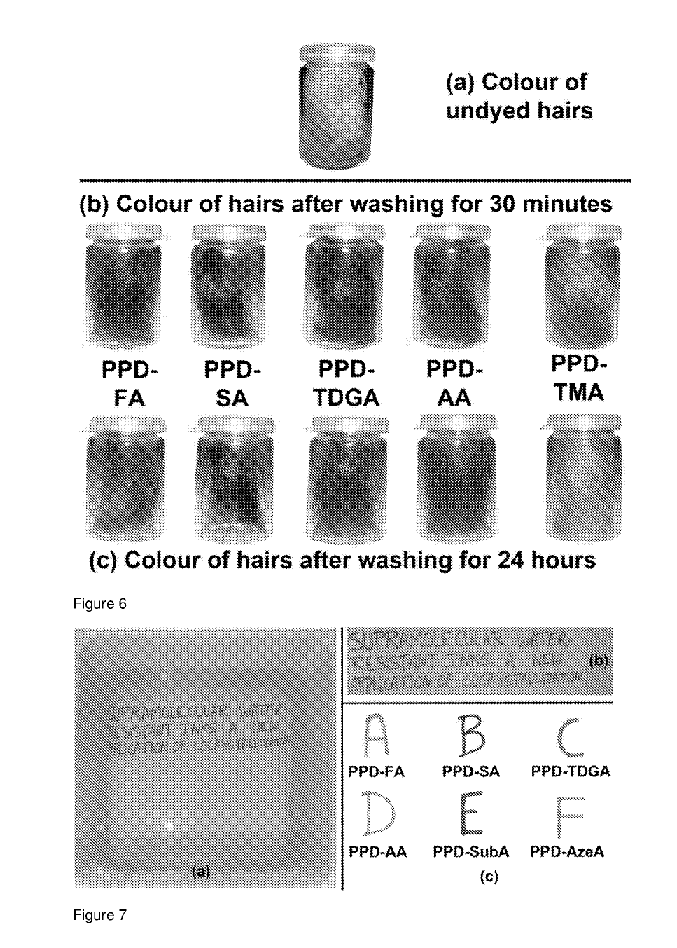

FIG. 6: (a) Undyed hairs. Dyed hairs after washing continuously in hot soapy solution for (b) 30 minutes and (c) 24 hours

FIG. 7 shows (a) Writing with PPD-SA ink immersed in hot water (70° C.) (b) Dried paper after 4 hour of hot water treatment (c) different coloured inks formed by cocrystallization between PPD and various acids; and

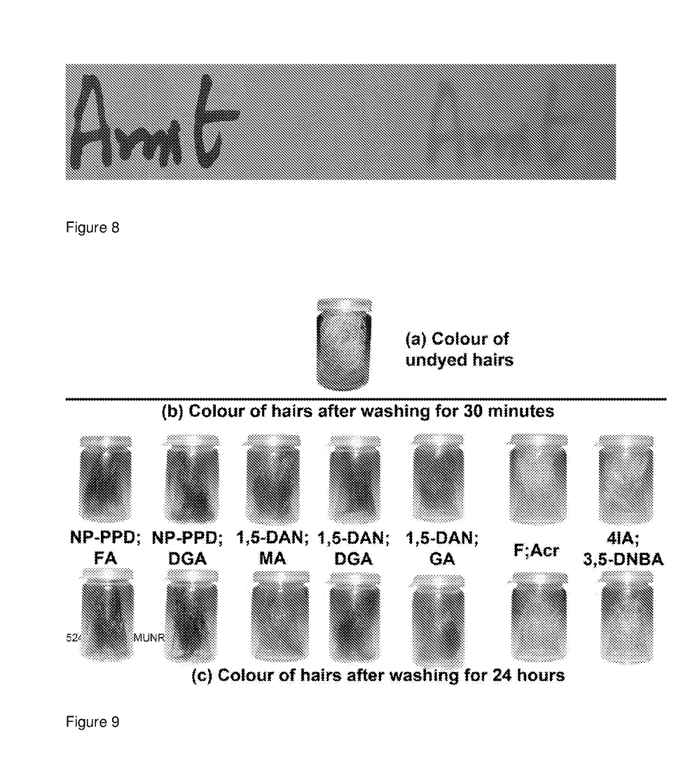

FIG. 8 shows the Deinking of paper by washing with methanol.

FIG. 9 (a) Undyed hairs. Dyed hairs after washing continuously in hot soapy solution for (b) 30 minutes and (c) 24 hours

### EXAMPLE 1: PPD/LAWSONE CO-CRYSTAL DEVELOPMENT

We developed a new type of PPD based hair dyes by cocrystallization. Lawsone (L) was chosen as coformer. Lawsone is an orange coloured molecule and is present in henna. Our choice of coformer was based upon the fact that lawsone molecules bind well with the hairs and because of it henna is widely used as a hair dye (it imparts orange colour to the hairs). Henna is not only popular as a hair dye, but is also widely used for making henna tattoos, especially on palms and feet. Worldwide use of henna over skin, and that too for centuries, indicate that lawsone molecule is safe even for topical application. The safety of henna was recently confirmed by SCCS.

An attempt was made to form an adduct between PPD (colourless when freshly prepared) and lawsone (yellow in colour) by dissolving them in 1:1 molar ratio in methanol solution. The formation of adduct was first indicated by change of colour (FIG. 1 (a)). The blackish-brown solution so obtained was kept for slow evaporation and it resulted in the formation of blackish-brown crystals in 3 days. The crystal structure analysis revealed the formation of salt in the 1:2 molar ratio between PPD and L (FIG. 1 (b)) in which the molecules of PPD and lawsone interacted with each other by N⁺—H•••O⁻ and N+—H•••O interactions (FIG. 1 (c)).

We attempted to form the adduct in phase pure form by liquid assisted grinding (LAG) of PPD and L in 1:2 for 60 minutes at 30 Hz in the presence of 50 μL of the methanol or by mixing their methanol solutions in the same ratio as that observed in the crystal structure. Comparison of the PXRD patterns of material obtained from LAG and solution crystallization with the simulated pattern of the crystal structure indicated that we were successful in obtaining the same adduct by both the techniques (FIG. 2a), which was further confirmed by IR, TGA and DSC. An attempt was made to scale up the production of PPDL adduct to 20 gm by LAG and solution crystallizations, which was successful.

### EXAMPLE 2: HAIR DYING ABILITY OF PPDL

The dye obtained from co-crystallisation reaction between PPD and L in 1:2 molar ratio from methanol solution was tested for its dying performance. We chose grey hairs, which are considered to be toughest to dye (FIG. 3(a)). Hairs were dyed by immersing roughly 1 gm of hairs in 5 ml of supersaturated hair dye solution for 30 minutes, which resulted in reddish-brown hairs. Such saturated solutions were found to be containing around 0.75 g of PPDL per 10 ml of the solution. Supersaturated solutions may be obtained by just letting the solution evaporate under room temperature. We employed rotavapor to speed up this process.

The excess of hair dye was removed with the help of hot water. Dyed hairs were initially tested for its performance by stirring in hot soapy water maintained at 50° C., for 30 minutes. During this time the soapy water was changed 5 times. The PPDL hair dye worked very well and retained the colour (FIG. 3 (b)). In view of the initial product performance, we tested PPDL hair dye performance under extreme washing conditions for a longer period. Thus, dyed hairs were washed for 24 hrs by stirring in hot soapy water maintained at 50° C. During this washing, we changed the soapy water some 20 times. The 24 hr. washing experiment demonstrated that the hair dye was retained even under these extreme washing conditions (FIG. 3 (c)).

It is widely believed that the dye molecules interact with hairs by hydrogen bonds. (see for example Morel, O. J. X.; Christie, R. M. Chemical Reviews 2011, 111, 2537) Without wishing to be bound by theory, it is noted that the PPDL hair dye is a salt, which can form stronger charge-assisted hydrogen bonds with the hairs, as compared to currently marked hair dyes which can only form neutral hydrogen bonds. It is also noteworthy that this dye works even without the ammonia. In the currently marketed PPD-based hair dye products ammonia is added to open up the pores of the hairs, so that the big hair dye molecules can enter into the hairs. Since the molecules used in PPDL hair dye are much smaller, it is thought that they can enter through smaller pores, avoiding the need for use of ammonia.

As can be seen, the present invention provides hair dyes which are ammonia free and do not require H₂O₂ for colour generation. The exemplified hair dye shows a great colour fastness, and the dyed hairs retained their colour even after washing in hot soapy water for 24 hrs. The significance of this new class of hair dyes is further enhanced, due to recent reports relating the currently marketed PPD-based oxidative hair dyes with cancer (if they come in topical contact with the skin http://ec.europa.eu/health/scientific_committees/consumer_safety/does/sccs_o_094.pdf.).

### EXAMPLE 3: CO-CRYSTALLISATION OF PPD WITH ORGANIC ACIDS

We cocrystallized PPD with various acids fumaric (FA), succinic (SA), thiodiglycolic (TDGA), adipic (AA), suberic (SubA) and azelaic (AzeA), sebacic (SebA), 1,3-phenylenediacetic, (1,3-PDAA), 1,4-phenylenediacetic, (1,4-PDAA) and trimesic (TMA) acids. In all the reactions, the formation of adduct was strongly indicated by colour change observed during all the cocrystallization experiments. A typical example demonstrating colour change observed during these cocrystallizations is as shown in FIG. 4. The cocrystallization reactions were initially carried out in 1:1 molar ratio between PPD and various acids from methanol solution. Single crystal X-ray diffraction measurements were taken and the molar ratio of PPD with various acids was confirmed. This ratio allowed synthesis of the products from the correct ratio of starting components.

All the co-crystals were structurally characterized using single crystal XRD. The analysis of their crystal structures revealed that all the co-crystallizations yielded salts by proton transfers from the acid to the PPD molecules (FIG. 5).

It has also been possible to make the various coloured cocrystals described above using a LAG route as mentioned in Example 1.

### EXAMPLE 4: USE OF PPD/ORGANIC ACID CO-CRYSTALS AS HAIR DYES AND INKS

The dyes obtained from co-crystallisation reaction between PPD and various acids were tested for their dying performance. We chose grey hairs, which are considered to be toughest to dye (FIG. 6(a)). Hairs were dyed by immersing roughly 1 gm of hairs in 5 m of saturated hair dye solution and allowing it to remain in contact with the hair until the desired hair color has been attained. The excess of hair dye was removed with the help of hot water. Dyed hairs were initially tested for its performance by stirring in hot soapy water maintained at 50° C., for 30 minutes. During this time the soapy water was changed 5 times. The hair dyes worked very well and retained the colour. The dying performance of some of these dyes is as shown in FIG. 6 (b). We then tested the hair dyes under extreme washing conditions for a longer period. Thus, dyed hairs were washed for 24 hrs by stirring in hot soapy water maintained at 50° C. During this washing, we changed the soapy water some 20 times. The 24 hr. washing experiment demonstrated that the hair dye was retained even under these extreme washing conditions (FIG. 6 (c)).

We explored the potential of these coloured solutions obtained during cocrystallization reactions as inks. For this purpose the solutions were concentrated, simply by evaporation under room temperature for 2 days. These solutions can be added to writing implements and used as inks. A point of note is that these inks are resistant to water despite being in an ionised form; even copious amounts of water failed to make any measurable impact in the spread of the ink. To test the resistivity of the ink we tested our inks by immersing the text in hot water (70° C.) for 4 hours (see FIG. 7 (a)). Very encouragingly, this new product proved to be water resistant (see FIG. 7 (b)) and we were able to obtain inks of different colours and shades, as demonstrated in FIG. 7 (c). It is noteworthy to mention most of the marketed inks are a complex mixture containing following 8 components: (1) Colourants, (2) solvent, (3) dispersants, (4) polymeric resins, (5) humectants (retard premature drying), (6) antiforming agents, (7) wetting agents and (8) pH modifiers. The colourant are usually inorganic or organic dyes or pigments molecules and costs around 50% of the cost of the ink. We carried out SEM analysis of the blank paper to have a better insight into its structural features. The analysis revealed that the main components of the paper were cellulose fibres and CaCO₃. CaCO₃ is routinely used by the paper industry, mainly to make it white. Analysis of paper and ink indicated the possibility of formation of strong hydrogen bonds between cellulose fibres and ink. Again without wishing to be bound by theory, we believe that due to ionic nature of the ink, it may also form some ionic interactions with the CaCO₃ present in the paper. It may be due to these two reasons that the ink binds strongly with the paper.

With the increase in the demand of the paper, there is a growing concern about the reclycing of paper. According to an estimate 1 tonne of recycled paper prevents around 17 mature trees from cutting. One way to achieve this goal is to design the inks which can be easily removed. We tested erasing our inks by simply washing the paper with methanol solvent. The results were very encouraging and the ink was removed to a great extent by briefly washing the paper with methanol (FIG. 8). The ease with which the ink has been removed is remarkable and very significant given the drive to more ecologically friendly means of manufacture and processing.

The above describes the ability of forming inks by cocrystallization reactions. The technology bypasses synthetic pathways for the generation of coloured molecules. The inks were smooth to use and showed a great water resistance. Analysis of the crystal structure of the co-crystals within the ink releaved a proton transfer from acids to the PPD molecules, forming salt. The strong interaction between ink and paper, which also led to water-resistant behaviour might be linked to the possibility of formation of strong hydrogen bonds between ink components and cellulose fibres, as well as the possibility of ionic interactions between ions of ink and CaCO₃.

### EXAMPLE 5: USE OF OTHER COLOURED CO-CRYSTALS AS HAIR DYES

In an analogous manner to Examples 3 and 4, further coloured co-crystals were prepared using various combinations of co-crystal forming molecules and the resulting coloured co-crystals tested for their hair dying ability.

Coloured co-crystals were formed from the following mixtures of molecules, with the molar ration indicated in brackets:
NPPPD;DGA=N-Phenyl-Para-Phenylene Diamine+Diglycolic acid (1:1)
NPPPD;FA=N-Phenyl-Para-Phenylene Diamine+Fumaric acid (1:1)
1,5-DAN;MA=1,5-Naphthalenediamine+Malonic acid (1:1)
1,5-DAN;DGA=1,5-Naphthalenediamine+Diglycolic acid (1:1)
1,5-DAN;GA=1,5-Naphthalenediamine+glutaric acid (1:2)

In addition we also tested the dyeing potential of already known coloured cocrystals e.g. of
F;Acr.=Fluorescene+Acridine (1:2)
4-IA;3,5-DNBA=4-lodoaniline+3,5-Dinitrobenzoic acid+methanol (2:2:1)

Solutions comprising the coloured co-crystals resulting from the above co-crystallisation reactions were used to dye hair and the results are shown in FIG. 9. As can be seen from FIG. 9, a variety of coloured co-crystal solutions, in addition to the PPD/FA solution described above, were made by the present inventors and tested for their hair-dying ability. These additional coloured co-crystal solutions were all capable of dying grey-hair in a variety of colours, ranging from yellow to brown/black. The F/Acr co-crystal solution even provided the dyed hair with a fluorescent property, which some users may find appealing. The various coloured co-crystal solutions also displayed significant colour fastness, as there was little de-colouration following washing.

Surface analysis of the dyed hair revealed no significant change in the surface properties of the hairs after dyeing indicating that these dyes are not harsh on the hair surface.

Scale-up: From an industrial prospective, scalability and ease of processing are very important factors. Considering this in mind, a cocrystal hair dye (PPD-SebA) was chosen for scale up, based upon the cost and availability of the chemicals. The system was scaled up to 275 g scale both by LAG and solution methods, as discussed herein. The materials obtained from LAG (pinkish-white) and solution crystallizations (dark bluish-black) differ in color, but PXRD analysis shows (data not shown) that the products of both these methods are the same.

Supramolecular hair dye formulation: After bulk preparation, we attempted to formulate the PPD-SebA cocrystal into a formulation that could be applied to hairs directly by potential customers. For this purpose, 4 ml of a concentrated solution of PPD-SebA in methanol was diluted with the addition of 2 ml of water to form a solution. To this 6 g of solid PPD-SebA (obtained from solution crystallization from methanol) was added to form a paste. During the paste formation the mixture was kneaded to break up the solid that had agglomerated. This paste was used to dye wet grey hairs (soaked in water for 2 minutes to swell the hair) for 30 minutes. The excess hair dye, which was adhering to the surface of the hairs, was easily recovered by rubbing the hairs between the finger tips. The dyed hairs were tested for their dying performance by washing them 25 times with soapy water and found to retain the colour. To investigate the reusability of the cocrystals for dying, 3 g of the recovered material was again converted to a paste by adding 1 ml of water and 2 ml of concentrated solution of PPD-SebA. Dyeing was done for 30 minutes, which was followed by 25 washings with soapy water. The dyeing performance of the recovered material was same as that of dye used for the first time. It is noteworthy to mention that conventional oxidative hair dyes are not reusable and any hair dye left after use is simply discarded.

Binding Forces: Hair dye performance is determined by the binding forces between hairs and dye. The dye molecules primarily bind the hairs using hydrogen bonds. Hair dye companies aim at increasing the strength of these hydrogen bonds for better product performance. The hair dyes presented herein are ionic and hence have the potential to form stronger charge assisted hydrogen bonds with the hairs in comparison to the neutral molecules formed via the oxidative reaction.

Potential of cocrystals to form a range of colors (color palette): One of the prime requirements of the hair dye industry is to produce a range of colors. The oxidative hair dye (OHD) industry relies on changing the dye precursor-coupler combination to achieve the same. The precursors and couplers are chosen in such a way that they can form the more conjugated colored end product. Unfortunately, OHD industry is limited in terms of the number of approved safe molecules (around 100) which they can use as dye precursors and couplers. The present hair dyes may have an advantage over OHDs in terms of the number of safe molecules (thousands of safe coformers for the hair dyes which can be chosen from EAFUSand GRAS lists).

1. (canceled)

2. A hair dye, ink and/or paint composition comprising a coloured co-crystal formed from a first and a second coformer.

3-14. (canceled)

15. The hair dye, ink and/or paint composition according to claim 2 wherein a first component is selected from the group consisting of: p-phenylenediamine (PPD) or a derivative thereof, N-Phenyl-Para-Phenylene Diamine, 1,5-Naphthalenediamine, 4-lodoaniline, 1-anilinonaphthalene-8-sulfonic acid, 1,3-Dichloro-7-hydroxy-9,9-dimethyl-2(9H)-acridinone, 1,4-bis-p-cyanostyrylbenzene, 2-((E)-4-((E)-4-cyanostyryl)styryl)benzonitrile, 2,2′4(1E, 1′E)-1,4-phenylenebis(ethene-2,1-diyl))dibenzonitrile, 2-Methylbenzoxazole, 2,7-dichlorofluorescein, 2″,7′-bis-(2-carboxyethyl)-5-(and-6)-carboxyfluorescein 2,5-diphenyloxazole, 4-Dimethylamino-4′-nitrostilbene, 5(6)-Carboxyfluorescein, 5(6)-Carboxynaphtofluorescein, 5(6)-Carboxytetramethylrhodamine B, 5-(and-6)-carboxy-2′,7′-dichlorofluorescein, 5-(N-hexadecanoyl)aminoeosin, 5-Chloromethylfluorescein, 5-carboxyfluorescein, 6,8-difluoro-7-hydroxy-4-methylcoumarin, 6-carboxyrhodamine 6G, 7-hydroxy-4-methylcoumarin, Acridine (orange and yellow), Alexa Fluor, Alcian yellow GXS, Alizarin, Alizarin red S, Alizarin yellow GG, Alizarin yellow R, Anthoxanthin, Arylide yellow, ATTO, Auramine O, Azophloxin, Azo compound, Bilin, Bismarck, Bistre, Bone char, brown R, Bismarck brown Y, Brilliant cresyl blue, Calcein, Caput mortuum, Carmine, Chrysoidine R, Chrysoidine Y, Coumarin, Congo red, Crimson, Crystal violet, Doxorubicin, Diarylide pigment, Dragon's blood, Fluorescein, Fuchsin acid, epicocconone, Gamboge, Gentian violet, Indian yellow, Indigo dye, Janus green, Lissamine fast yellow, Marina Blue, Martius yellow, Meldola blue, Metanil yellow, Methyl orange, Methyl red, Methylene Blue, Monobromobimane, Monochlorobimane, Naphthalene black 12B, Naphthofluorescein, Naphthol green B, Naphthol yellow S, Naphthol Red, nile blue, nile red, Oxazin, Ommochrome, Orange G, Perinone, Phthalocyanine Blue BN, Phthalocyanine Green G, Pigment Yellow (10,16, 81, 83), Piroxicam, Quinacridone, Riboflavin, Rose bengal, Rose madder, Rylene dye, Sepia (color), Sudan II, Sulforhodamine, Titan yellow, Tyrian purple, Tropaeolin O, Tropaeolin OO, Tropaeolin OOO, Victoria blue 4R, Victoria blue B, Victoria blue R, and Xylene cyanol FF.

16. The hair dye, ink and/or paint composition according to claim 2 wherein the second coformer is selected from the group consisting of: carboxylic acid, alcohol, amines, aldehydes, nitro, nitroso, ketones, ethers, esters, amides, acetals, ketals, imide, nitrile, isonitrile, halo, acylhalide, nitroso, pyridine, sulfoxide, sulfinic acid, sulphonic acids, cyanates, isocyanates, thiols, thial, thione, thiocyanates, isothiocyanates, semicarbazones, thiosemicarbazones, ureas, thioureas, amides, phosphonic acids, phosphine, phosphate, phosphodiester, boronic acids, boronic esters, borinic acids and borinic esters functionalities.

17. The hair dye, ink and/or paint composition according to claim 16 wherein the second coformer is selected from the group consisting of aliphatic and aromatic mono, di or polycarboxylic acids and alcohols of variable chain lengths (e.g acetic acid, oxalic acid, malonic acid, maleic acid, lactic acid, tartaric acid, citric acid, fumaric acid, succinic acid, acetylenedicarboxylic acid, mesaconic acid, trans-acotinic acid, thiodiglycolic acid, diglycolic acid, glutaric acid, adipic acid, hexanoic acid, pimelic acid, suberic acid, octanoic acid, azelaic acid, sebacic acid, decanoic acid, undecanedioic, dodecanedioic, oleic acid, arachidic acid, stearic acid, palmitic acid, erucic acid, arachidonic acid, linoleic acid, linolenic acid, phthalic acid, isophthalic acid, terephthalic acid, benzenetricarboxylic acids, benzenetetrracarboxylic acids, benzenepentacarboxylic acids, benzenehexacarboxylic acid, disubstituted benzoic acids 3,5 dinitrobenzoic acid, phenylenediacetic acids, phenols, benzenediol, benzenetriols, benzenetetraols, benzenepentaols, benzenehexaols, and aliphatic alcohols containg one of more hydroxyl groups.

18. The hair dye, ink and/or paint composition according to claim 17 wherein the second coformer is selected from the group consisting of alcohols and ketones include flavonoids, such as flavones, flavanols, isoflavones, chalcones and catechins; terenoids and isoprenoids; naphthoquinones and anthraquinones; alkaloids, naphthoquinones and anthraquinones, naphthoquinone, juglone, lawsone, alkannin, anthraquinone and alizarin.

19. The hair dye, ink and/or paint composition according to claim 18 wherein the second coformer is Lawsone.

20. A coloured co-crystal for use as a hair dye formed from p-phenylenediamine (PPD) or a derivative thereof in combination with an alcohol or ketone.

21. The coloured co-crystal according to claim 20, wherein the alcohol or ketone is selected from the group consisting of flavonoids, flavones, flavanols, isoflavones, chalcones, catechins, terenoids, isoprenoids, naphthoquinones, anthraquinones and alkanoids.

22. The coloured co-crystal according to claim 20 wherein the alcohol or ketone is selected from the group consisting of naphthoquinones, anthraquinones, naphthoquinone, juglone, lawsone, alkannin, anthraquinone and alizarin.

23. The coloured co-crystals according to claim 20 formed from PPD or a derivative thereof and lawsone.

24. A coloured co-crystal for use as a hair dye formed from PPD, or a derivative cocrystallized with fumaric (FA), succinic (SA), glycolic, malonic, glutaric, thiodiglycolic (TDGA), adipic (AA), suberic (SubA) and azelaic (AzeA), sebacic (SebA), 1,3-phenylenediacetic, (1,3-PDAA), 1,4-phenylenediacetic, (1,4-PDAA) or trimesic (TMA) acids.

25. The hair dye, ink and/or paint composition according to claim 2, or coloured co-crystal according to claim 20, wherein the PPD derivative is selected from the group consisting of: p-toluenediamine; 2-chloro-p-phenylenediamine; N-phenyl-p-phenylenediamine; N-2-methoxyethyl-p-phenylenediamine; N,N-bis-hydroxyethyl-p-phenylenediamine; 2-hydroxymethyl-p-phenylenediamine; 2-hydroxyethyl-p-phenylenediamine; 4,4′-diaminodiphenylamine; 2,6-dimethyl-p-phenylenediamine; 2-isopropyl-p-phenylenediamine; N-(2-hydroxypropyl)-p-phenylenediamine; 2-propyl-p-phenylenediamine; 1,3-bis-(N-hydroxyethyl)-N-(4-aminophenyl)amino)-2-propanol; and 2-methyl-4-dimethylaminoaniline.

26. A hair dye composition comprising a coloured co-crystal according to claim 20 or cocrystals of fluorescein and acridine, or a methanol solvate of 4-iodoanailine and 3,5 dinitrobenzoic acid, together with one or more hair dye formulation components.

27. A hair dye composition according to claim 26, wherein the one or more hair dye components is a wetting agent, emulsifying agent, surfactant, thickener, conditioner, pH adjuster, lanolin or lanolin derivative, cholesterol, pantothenic acid, an adjuvant or cosmetic additive.

28. A preformed hair dye solution comprising a formulation according to claim 2.

29. The coloured co-crystal according to claim 20, wherein the PPD derivative is selected from the group consisting of: p-toluenediamine; 2-chloro-p-phenylenediamine; N-phenyl-p-phenylenediamine; N-2-methoxyethyl-p-phenylenediamine; N,N-bis-hydroxyethyl-p-phenylenediamine; 2-hydroxymethyl-p-phenylenediamine; 2-hydroxyethyl-p-phenylenediamine; 4,4′-diaminodiphenylamine; 2,6-dimethyl-p-phenylenediamine; 2-isopropyl-p-phenylenediamine; N-(2-hydroxypropyl)-p-phenylenediamine; 2-propyl-p-phenylenediamine; 1,3-bis-(N-hydroxyethyl)-N-(4-aminophenyl)amino)-2-propanol; and 2-methyl-4-dimethylamino aniline.
